SEQUENCE LISTING (4 sequences)

SEQ ID NO: 1 - protein, 17 aa, synthetic construct
YIDTNNDGWYEGDELLA

SEQ ID NO: 2 - protein, 20 aa, synthetic construct
FIDTNNDGWIEGDELLLEEG

SEQ ID NO: 3 - protein, 18 aa, synthetic construct
  MOD_RES 8: note = FITC
YIDTNNDKGWYEGDELLA

SEQ ID NO: 4 - protein, 133 aa, Methylorubrum extorquens
MAFRLSSAVLLAALVAAPAYAAPTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIR

(19) United States
(12) Patent Application Publication
(10) Pub. No.: US 2024/0400719 A1
(43) Pub. Date: Dec. 5, 2024

(54) MATERIALS FOR VALUABLE METAL RECOVERY

(71) Applicant: Trustees of Tufts College, Medford, MA (US)

(72) Inventors: David L. Kaplan, Concord, MA (US); Huan-Hsuan Hsu, Pittsford, NY (US); Ryan Scheel, Syracuse, NY (US); Xiaocheng Jiang, Zhejiang (CN)

(21) Appl. No.: 18/652,036

(22) Filed: May 1, 2024

Related U.S. Application Data
(63) Continuation of application No. PCT/US22/79408, filed on Nov. 7, 2022.
(60) Provisional application No. 63/263,648, filed on Nov. 5, 2021.

(51) Int. Cl. C07K 16/44; B01D 67/00; B01D 69/02; B01D 69/10; B01D 69/12; B01D 69/14; B01D 71/74; C07K 14/435; G01N 21/78

(52) U.S. Cl. CPC C07K 16/44; B01D 67/00046; B01D 69/02; B01D 69/107; B01D 69/1216; B01D 69/144; B01D 71/74; C07K 14/43586; B01D 2323/12; B01D 2325/02833; B01D 2325/04; C07K 2317/33; C07K 2317/92; C07K 2319/00; G01N 21/78

(57) ABSTRACT

Capture membranes for lanthanide metal ions can be made from fusion proteins having at least one lanthanide metal binding sequence (SEQ ID NO: 1-4) covalently bound to a silk-elastin-like polymer (SELP). Capture membranes can be made from silk nanofibrils that are surface-modified with a lanthanide metal binding molecule. The capture membranes can have a layered structure or can contained cross-linked peptides in a hydrogel.

### CROSS-REFERENCE TO RELATED APPLICATIONS

This application is a continuation of International Application Serial Number PCT/US2022/079408, filed Nov. 7, 2022. International Application Serial Number PCT/US2022/079408 is related to and claims priority to U.S. Provisional Patent Application No. 63/263,648, filed Nov. 5, 2021. Each of the foregoing patent applications is incorporated herein by reference in their entirety for all purposes.

### STATEMENT REGARDING FEDERALLY SPONSORED RESEARCH

This invention was made with government support under Grant DE-AR0001339 awarded by the Advanced Research Project Agency—Energy (ARPA-e, Department of Energy). The government has certain rights in the invention.

### REFERENCE TO A SEQUENCE LISTING

A Sequence Listing accompanies this application and is submitted as an xml file of the sequence listing named “166118_01259.xml” which is 5,055 bytes in size and was created on Nov. 7, 2022. The sequence listing is electronically submitted via Patent Center and is incorporated herein by reference in its entirety.

### FIELD OF THE INVENTION

The disclosed technology is generally directed to metal recovery. More particularly the technology is directed to metal recovery using silk-based materials.

### BACKGROUND OF THE INVENTION

Lanthanide waste arising from growth in the mining, production and use of lanthanide-containing materials correlates to an increased need to develop efficient methods of removing these metals from waste streams. Since rare earth elements are toxic to humans and other organisms, it is an important challenge to process the aqueous waste contaminated with these elements. As lanthanide-based materials are gaining importance in numerous commercial products such as electroluminescent devices, catalysts, fiber optic lasers, and fuel cells; waste streams arising from both the production and disposal of these products need to be addressed. Previous work has focused on ligand design, extraction studies, or chromatography.

### BRIEF SUMMARY OF THE INVENTION

A recombinant fusion protein can have at least one lanthanide metal binding sequence (SEQ ID NO: 1-4) covalently bound to a silk-elastin-like polymer (SELP), where the recombinant fusion protein exhibits an apparent or true binding affinity towards a lanthanide metal ion of between 2.0×10⁵ M⁻¹ and 1.8×10⁷ M⁻¹ or higher.

A modified silk nanofibril can have a lanthanide metal binding molecule coupled to a surface of a silk nanofibril, where the lanthanide metal binding molecule comprises at least one lanthanide metal binding sequence (SEQ ID NO: 1-4).

A method of producing a metal-binding silk protein following the steps of (i) expressing in a host cell the lanthanide-metal-binding silk protein comprising at least one metal-binding sequence and at least one SELP sequence, where the metal-binding sequence is one of SEQ ID NO: 1-4, and (ii) purifying the lanthanide metal-binding silk protein.

A modified silk protein having (i) at least one modified lanthanide metal-binding protein according to SEQ ID NO: 1-4, or a variant thereof which differs in each case from said sequence by 1 amino acid substitution, 1 to 2 amino acid deletion(s), and/or 1 to 2 amino acid insertion(s), and (ii) at least one modified SELP according to claim 2, or a variant thereof which differs in each case from said sequence by 1 amino acid substitution, 1 to 2 amino acid deletion(s), and/or 1 to 2 amino acid insertion(s).

A method of creating a layered membrane according to the following steps: (i) a first dispersion of a silk nanofibril is filtered over a porous polycarbonate membrane substrate (pore size: 0.2 m) using vacuum filtration to form a first layer; and (ii) a second dispersion of the fusion protein of claim 1 is vacuum filtered over the first layer to create a second layer, wherein the first dispersion and second dispersion each have a protein concentration between 0.1% and 1.0%, weight by volume.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. A) Schematic of chemical functionalization of LBT-2 onto the SNFs through approach-1. In brief, the carboxylic acid moieties of SNFs (Asp, Glu) were carbodiimide coupled with the N-terminus free amines of LBTs in the presence of N-(3-Dimethylaminopropyl)-N′-ethyl carbodiimide (EDC) and N-Hydroxy Succinimide (NHS) at pH 6 for 18 h at room temperature (RT). B, C) represents Scanning Electron Microscopy (SEM) images of LBT-2 (FIDTNNDGWIEGDELLLEEG) modified SNFs by carbodiimide coupling as described in (A). SEM images demonstrate retention of fibrillar morphology of SNFs post LBT-2 modification. D) Schematic of chemical functionalization of LBT-1 onto the SNFs through approach-2. In brief, the phenolic moieties of SNFs (Tyr) were diazonium coupled with 4-amino benzoic acid to increase the carboxylic acid content, which were carbodiimide coupled with the N-terminus free amines of LBTs in the presence of EDC and NHS at pH 6 for 18 h at room temperature. E, F) represents SEM images of LBT-1 (YIDTNNDGWYEGDELLA) modified SNFs by Approach-2 as described in (D).

FIG. 2. A) Schematic of chemical functionalization of LBT(FITC) onto the SNFs using carbodiimide coupling. In brief, the carboxylic acid moieties of SNFs (Asp, Glu) were carbodiimide coupled with the N-terminus free amines of LBT(FITC) in the presence of N-(3-Dimethylaminopropyl)-N′-ethyl carbodiimide (EDC) and N-Hydroxy Succinimide (NHS) at pH 6 for 18 h at room temperature (RT). B) Quantification of LBT(FITC) functionalized onto SNFs was performed using fluorescence spectroscopy and calibration curve fitting based on the data shown in panels C and D. C) Fluorescence spectrum of SNF modified with LBT(FITC), with the emission maximum at 520 nm used for further calculation. D) Fluorescence spectra for varying concentrations of pure LBT(FITC) used to generate the calibration curve in panel B.

FIG. 3. A) Luminescence spectra of LBT-1 solution (100 PM, line #1), TbCl3 solution (100 μM, line #3) and mixed solutions of LBT-1 (100 μM) & TbCl3 (100 μM), λex=280 nm (line #2), B) luminescence spectra of TbCl3 solution (333 μM) in presence of LBT-1 functionalized SNF dispersion (0.025%, w/v) through carbodiimide coupling (Approach-1, SNF(D, E)-LBT-1) and diazonium & carbodiimide coupling (Approach-2, SNF(Y)-LBT-1), and unmodified SNF dispersion (0.025%, w/v), λex=280 nm. The characteristic luminescence peaks of Tb+3 (490, 545, 585, and 620 nm) were observed in presence of LBT modified SNFs from both approaches (approach-1 line #1 and approach-2 line #2) validating the chelation of Tb+3 with LBT modified SNFs. Unmodified SNFs also demonstrates similar luminescence behavior (line #3) with significantly lower intensity.

FIG. 4. Completed genetic constructs for LBT-SELP fusion proteins. Amino acid sequences of the Tag and SELP components are shown in the inset panel. The complete genetic library contains 39 different constructs due to the 3 SELP sequences, the 4 separate tags, and the 4 different configurations (no-, low-, medium-, and high-binding).

FIG. 5. Example of SDS-PAGE analysis of pure LBT2(S₂E_{8V})₈. The single band between 30 and 40 kDa is indicative of pure LBT-SELP (˜37 kDa expected). The lyophilized protein is shown on the right.

FIG. 6. SDS-PAGE analysis of LBT1₄(S₂E_{8C})₈ subjected to ammonium sulfate precipitation. L denotes the crude lysate prior to addition of ammonium sulfate, and the bands of the target protein are outlined with a box.

FIG. 7. ¹H-NMR spectrum of LBT2(S₂E_{8V})₈ dissolved in deuterated dimethyl sulfoxide (DMSO). The peak assignments between 4.5 and 0.5 ppm are indicative of the SELP component of the protein, including the small chemical shift (σ) at 1.05 from the γ-protons of threonine (Thr) which is present between the SELP monomers as an artifact of the concatemerization process. The expanded region between 7.5 and 6.8 ppm highlights the chemical shifts indicative of the aromatic residues phenylalanine and tryptophan which are only present in the lanthanide-binding tag.

FIG. 8. ¹H-NMR spectra of low-binding SELP LBT1-(S₂E_{8C})₈ (bottom spectrum) and medium-binding LBT1₄-(S₂E_{8C})₈ (top spectrum) dissolved in deuterated dimethyl sulfoxide (DMSO). The peaks between 4.5 and 0.5 ppm are indicative of the SELP component of the protein, including the small chemical shift (σ) at 1.05 from the γ-protons of threonine (Thr) which is present between the SELP monomers as an artifact of the concatemerization process. The highlighted region between 7.5 and 6.8 ppm contains the chemical shifts indicative of the aromatic residues tyrosine and tryptophan which are only present in the lanthanide-binding tag, whose intensity increases ˜4-fold from the low-binding to the medium-binding SELP. Integration of the valine peak (σ=0.8) was normalized to 1 to compare the intensity of the aromatic peaks.

FIG. 9. Tb³⁺ titration of LBT1-(S₂E_{8Y})₈ and LBT1-(S₂E_{8Y})₈, labelled L1Y8 and L2Y8, respectively. Fluorescence intensity was measured with a λₑₓ/λₑₘ of 280/544 nm, and log-logistic regression performed using the drc package in R.

FIG. 10. Competition binding experiments with low-binding SELPs in the presence of 10 μM Tb³⁺ and varying concentrations of competitor metal ions. SELPs are named by their binding tag (H=His×10, V=V5, L1=LBT1, L2=LBT2) followed by the single letter amino acid code of the guest residue for that SELP (C=cysteine, V=valine, Y=tyrosine). Data is an average of triplicate experiments fit with a log-logistic regression line.

FIG. 11. A) Schematic of different steps associated with the preparation of SNF membranes; i) SNF was chemically functionalized with LBTs through diazonium and/or carbodiimide coupling; ii) certain amount of SNF or SNF-LBT dispersion was vacuum filtered to form SNF or SNF-LBT membranes under 70 KPa vacuum pressure with a porous polycarbonate supporting membrane (pore size 0.2 micron). B, C, D, E) SEM images of cross-sections of pure SNF membranes with fibrillar structures, the membrane thickness was controlled by the volumes of filtered SNF dispersion (porous polycarbonate membrane substrate shown in B&C, beneath the SNF membrane).

FIG. 12. A) Schematic of the steps associated with the preparation of SNF membranes using the direct filtering approach. SNFs were functionalized with LBT1 using diazonium and carbodiimide coupling (labelled as SNF(Y)LBT-1; Asp, Glu, and Tyr residues modified), and the aqueous dispersion vacuum filtered under 70 KPa vacuum pressure through a porous polycarbonate support membrane (0.2 μm pore size). B) Schematic of the steps to prepare SNF-LBT “sandwich” membranes; 1) unmodified SNF solution (1 mL, 0.1%) was used to form a thin membrane via the direct filtering approach, 2) SNF(Y)LBT-1 solution (5 mL, 0.15%) was vacuum filtered on top of the SNF membrane, 3) unmodified SNF solution was used again to form a thin capping membrane. C) Photos of SNF(Y)LBT-1 membranes formed via direct filtering (top) and “sandwich” (bottom), with direct filtering resulting in negligible deposition of material.

FIG. 13. SEM images of SNF membranes formed using the sandwich method, with leftmost images depicting the membrane surface, followed by two cross-sectional images at two magnifications on the right. A) A control membrane was formed via filtration of 2 consecutive aliquots of 1 mL SNF solution (0.1%). B) A multilayer SNF membrane with a central layer of functionalized SNF(Y)LBT-1; the approximate location of this layer has been outlined with a box based on the increase in thickness compared to Panel A. C) A multilayer SNF membrane with a central layer of SELP-LBT-2, indicated by the arrow.

FIG. 14. Schematic of membrane formation by SNF-SELP self-assembly for REE filtration.

FIG. 15. Representative SEM cross-section of self-assembled SNF-SELP membrane (24 hours, 37° C.). The average membrane thickness was 3±0.2 μm.

FIG. 16. ICP-OES analysis of Tb³⁺ filtrate. (A) Tb³⁺ remaining in solution after filtration, with unfiltered starting solution for reference. (B) Percent of Tb³⁺ captured, calculated from (A) as the inverse of Tb³⁺ concentration normalized to the starting concentration. (C) Tb³⁺ remaining in solution after filtration of combined Pd²⁺ and Tb³⁺ solution (100 ppm and 10 μM, respectively).

FIG. 17. ICP-OES analysis of Tb³⁺ filtrate after passage through SNF/L2Y₈ membrane, shown as the percent of total available Tb³⁺ captured.

FIG. 18. (A) ICP-OES analysis of Tb³⁺ filtrate after passage through SNF/SELP membranes (low, L2Y₈; medium, L2₄Y₈; high, L2₈Y₈), including (B) an analysis of SELP remaining in the flow-through. (C) Representative photo of the membranes on a UV lightbox showing fluorescence.

FIG. 19. Composite ICP-OES data from experiments (low, L2Y₈; medium, L2₄Y₈; high, L2₈Y₈). Data shown as the percent of total available Tb³⁺ captured.

FIG. 20. Schematic of membrane formation using cross-linked SELP hydrogels for REE filtration.

FIG. 21. ICP-OES analysis of Tb³⁺ filtrate after passage through SNF/SELP membranes. (A) Comparison of terbium captured (%) vs concentration of L2₄Y₈ SELP membranes, incubated at either 4° C. (blue) or 37° C. (orange). (B) Comparison of terbium captured vs SELP concentration (med, L2₄Y₈; high, L2₈Y₈).

### DETAILED DESCRIPTION OF THE INVENTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise. It should be appreciated that aspects of the disclosure that are described with respect to a system are applicable to the methods, and vice versa, unless the context explicitly dictates otherwise.

As disclosed herein, silk-based capture membranes can be engineered for the selective capture of Rare Earth Elements (REE) such as lanthanides. These membranes can be composed of silk nanofibrils (SNFs) that are chemically modified to contain covalently linked REE-binding peptides or lanthanide binding tags (LBT). Alternatively, the membranes can include bioengineered silk-elastin-like proteins (SELPs) with genetically encoded REE-binding peptide sequences for greater control and higher density of binding peptides. These membranes are efficient and selective for REE recovery. They can be reused to reduce material and energy consumption involved with the recovery of REEs from waste streams.

SNF-based capture membranes offer many advantageous features for metal recovery, including strong mechanical strength, tunable permeabilities, hydrolytic stability, and biodegradability. Single SNFs have a necklace-like morphology, as they are 3-4 nm in height, 30-40 nm in width, and up to 5 μm in length. SNF capture membranes can be readily prepared by filtering an SNF dispersion over a porous substrate using vacuum filtration. The SNFs entwine, assemble and remain on top of the porous substrate as the water passes through.

SELPs are based on a peptide having a silk sequence “S” and an elastin sequence “E”. These two sequences are alternating and repeating. S has the amino acid sequence (GAGAGS). E can be somewhat variable and can have one or more of the following amino acid sequences, for example (GAGAGY), (GVGVP), (GCGVP), or (GYGVP). Overall, the SELPs can have repeating units of S and E, for example (S₂E₈)₂ or (S₂E₈)₈.

As a component of the capture membranes disclosed herein, LBTs can have any of the sequences IDs 1-4 as listed below. This list is not meant to be limiting, and LBTs can have any amino acid sequence or molecular structure that is known to selectively bind REEs under normal conditions. LBTs are a group of peptides containing fewer than 25 amino acids that can tightly and selectively complex trivalent lanthanide ions and sensitize their fluorescence. It is contemplated that for efficacy in metal ion removal applications, LBTs can have an apparent or true binding affinity of between 10⁵ M⁻¹ and 10⁷ M⁻¹ or higher. LBT-2 has a lower dissociation constant (19 nM) towards terbium than LBT-1, which can correspond to a higher binding efficiency. Additionally, and alternatively, the 133-amino acid peptide lanmodulin (J. Am. Chem. Soc. 2018, 140, 44, 15056-15061 https://doi.org/10.1021/jacs.8b09842), which has a picomolar affinity for lanthanide ions, or a 99% portion thereof, or a 98% portion thereof, or a 97% portion thereof or a 96% portion, thereof or a 95% portion thereof, can be coupled to the SNF surface to be used as an LBT. A list of the LBT sequences is given in Table 1 below.

Further non-limiting examples of LBTs include linear ligands with multiple carboxylic acid groups such as ethylenediaminetetraacetic acid and pentetic acid, macrocyclic ligands with multiple carboxylic acid groups such as tetraxetan, and derivatives thereof. Additionally, LBTs can bear spectroscopic tags, for example FITC, for tracking and detection of the peptides.

TABLE 1
List of sequences and corresponding SEQ ID NO:
and abbreviations used herein.

| SEQ ID NO | Amino acid sequence | abbreviation |
|---|---|---|
| SEQ ID NO: 1 | YIDTNNDGWYEGDELLA | LBT1 |
| SEQ ID NO: 2 | FIDTNNDGWIEGDELLLEEG | LBT2 |
| SEQ ID NO: 3 | YIDTNNDK(FITC) | LBT1- |
| | GWYEGDELLA | FITC |
| SEQ ID NO: 4 | MAFRLSSAVL LAALVAAPAY | lanmodulin |
| | AAPTTTTKVD IAAFDPDKDG | |
| | TIDLKEALAA GSAAFDKLDP | |
| | DKDGTLDAKE LKGRVSEADL | |
| | KKLDPDNDGT LDKKEYLAAV | |
| | EAQFKAANPD NDGTIDAREL | |
| | ASPAGSALVN LIR | |

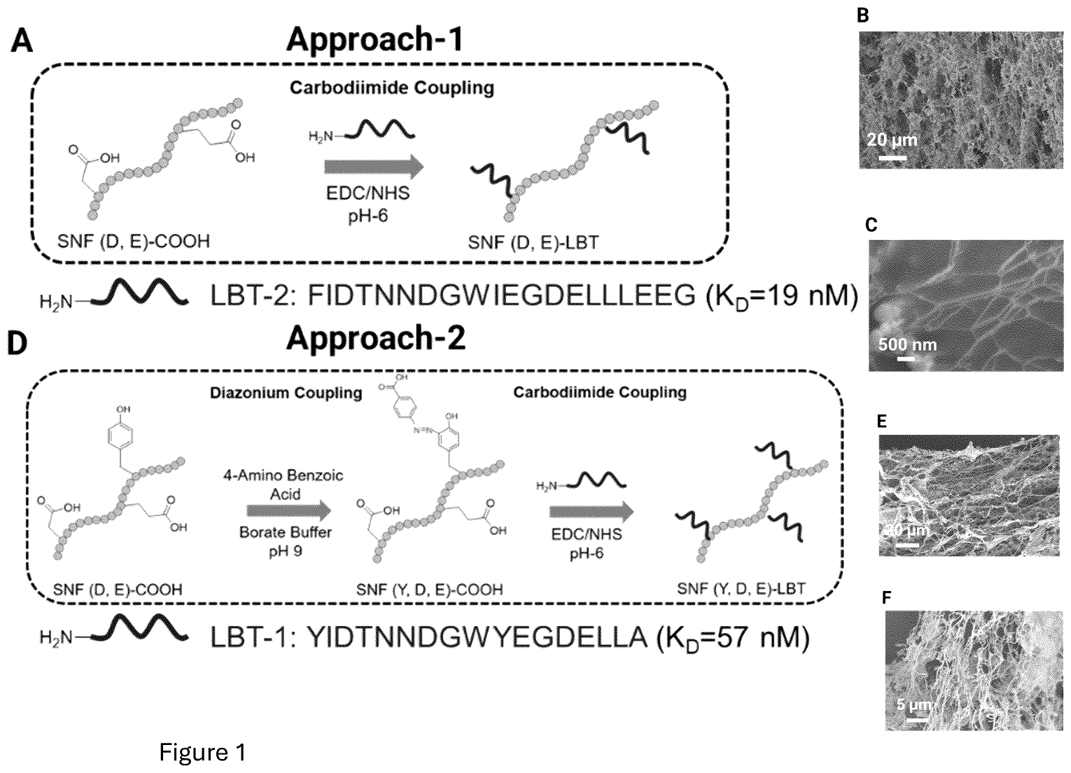

As SNFs are protein based and as such have exposed amino acid residues on the surface, chemical coupling can be utilized to attach an LBT to the surface of the SNFs. As shown in FIGS. 1A and 1D, the chemical functionalization of SNFs can be achieved through Approach-1, peptide coupling. In this example, existing amino acid residues are utilized to form a peptide bond between the terminal amine group of the LBT and free carboxylic acids on the SNF. For example, aspartic and glutamic acid residues can be peptide coupled with the LBT amine group using a carbodiimide approach. SNFs functionalized in this way with LBT-1 are referred to herein as SNF(D,E)LBT-1. Other coupling techniques known to be facile in mild aqueous conditions are also contemplated for coupling LBTs to the SNFs such as click chemistry, copper-free click chemistry, thiol-ene click chemistry, and other cross-coupling methods.

To maximize the number of LBT or metal binding sites for metal recovery, the number of LBTs attached SNFs should be maximized. As SNFs have a limited number of free carboxylic acids on the surface, one approach is to increase the number of carboxylic acids so that the number of attached LBT peptides can also be increased. In one example, shown as Approach-2 in FIG. 1D, tyrosine moieties (˜5.3 mol %) can be targeted by diazonium coupling for the addition of further carboxyl residues to the SNFs. Once the tyrosines have been modified, carbodiimide coupling of LBTs to the existing carboxyl residue in SNFs and new carboxyl groups generated by diazonium coupling of the tyrosine residues can occur. SNFs functionalized in this way with LBT-1 are referred to herein as SNF(Y)LBT-1.

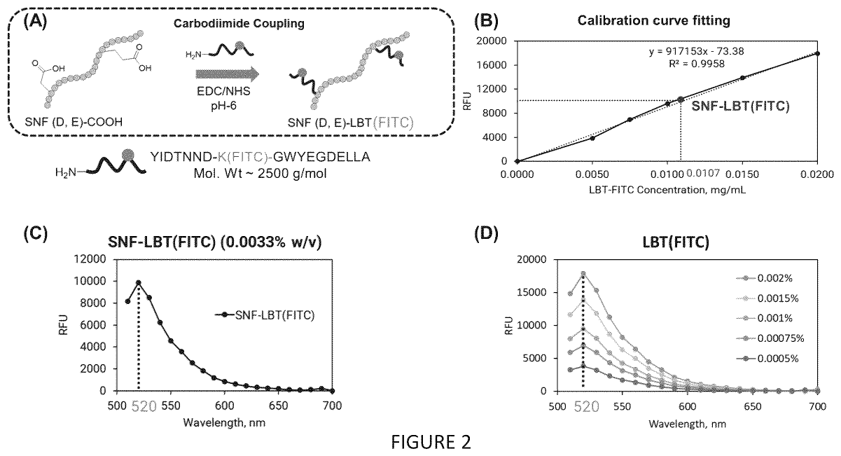

The SNFs largely retain their nanosized fibrillar structures after LBT functionalization by both approaches as shown by SEM (FIG. 1.1 B, C, E, F). The LBT functionalization of SNFs can further be validated by fluorescence spectroscopy and microscopy of the SNF functionalized by LBT-1 (FITC) as shown in FIG. 2.

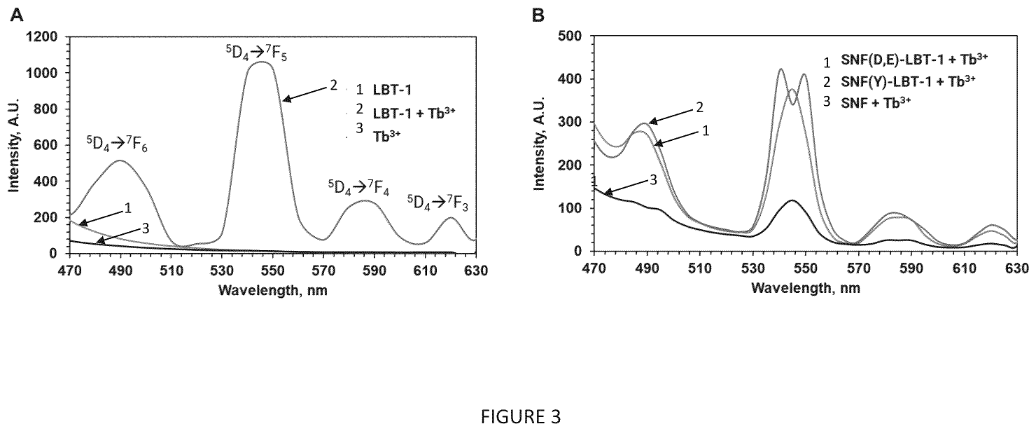

LBTs attached to SNFs are capable of chelating lanthanides. The coupling reactions that anchor the LBT to the SNF do not interfere with the chelation. For example, as shown in FIG. 3, the characteristic luminescence peaks of Tb³⁺ are clearly observed in solutions of Tb³⁺ with LBT-functionalized SNFs. For comparison, a TbCl₃ solution with LBT-1 shows the same peaks characteristic of chelated Tb³⁺.

### Construction and Expression of LBT-SELP Fusion Constructs

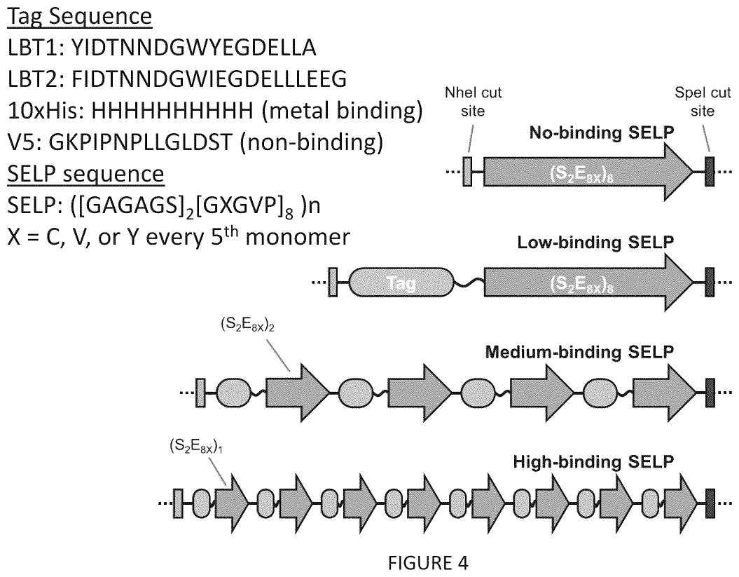

Silk-elastin-like proteins (SELPs) with genetically-encoded REE-binding peptide sequences can be bioengineered for greater control and higher density of binding peptides than can be achieved by chemical functionalization. In other words, a fusion protein combining the silk-elastin sequence and properties with the LBT sequence and properties can be designed to take advantage of both types of peptide. In one example, SELPs can have the amino acid sequence ((GAGAGS)ₐ(GXGVP)_{b})_{c}, wherein a ratio of a to b is between 1:3 and 1:5, wherein a is 1, 2, or 3, wherein c is between 5 and 20, and wherein X is valine, cysteine, or tyrosine. Concatemerization using DNA having multiple copies of the LBT and SELP sequences linked in series allows a variety of metal binding fusion proteins to be designed. For example, a series of SELP sequences with variable numbers of LBT sequences were designed. A first exemplary series includes a “low-binding” SELP with one LBT and one SELP sequence, a “medium binding” SELP with four LBT sequences alternating with four SELP sequences, and a “high-binding” SELP with eight LBT sequences alternating with eight SELP sequences. This series of SELP-LBT fusion proteins is illustrated in FIG. 4. Alternative sequences are contemplated, such as varying the number and grouping of SELP and LBT sequences (e.g., multiple consecutive LBT sequences followed by SELP), or by changing the ratio of silk and elastin blocks within the SELP sequence.

Synthetic oligonucleotides encoding for two lanthanide binding tags, LBT1 and LBT2, were designed with two sets of flanking restriction enzymes, an upstream NdeI followed by NheI, and a downstream SpeI followed by SacI. The outer set of enzymes (NdeI and SacI) were used to clone the lanthanide binding tags into a modified pET25 vector (pET25b[+] with the N-terminal His₆ tag removed). The new vectors, pET25-LBT1 and pET25-LBT2, were used to construct a set of LBT-SELP fusion constructs by ligating them with synthetic SELP monomer genes, (S₂E_{8X})₁. The generated constructs pET25-LBT1-(S₂E_{8X})₁ and pET25-LBT2-(S₂E_{8X})₁ were digested to identify the LBT-(S₂E_{8X})₁ fragments, which were excised, purified and ligated into the original pET25-LBT1-(S₂E_{8X})₁ to yield pET25-LBT12-(S₂E_{8X})₂. This concatemerization strategy can be repeated. For example, repeating twice more generates the pET25-LBT1₄-(S₂E_{8X})₄ expression vector, and repeating again generates the pET25-LBT1₈-(S₂E_{8X})₈ expression vector. Thus a library of LBT-SELP expression vectors was created with a set of “low-”, “medium-”, and “high-” binding constructs that contain 1, 2, 4 and 8 LBT sequences (LBT1 or LBT2) and 1, 2, 4, or 8 SELP monomers (S₂E_{8C}, S₂E_{8V}, or S₂E_{8Y}) in an alternating fashion. (FIG. 4) A similar strategy was used to create control constructs with either a non-metal binding V5 tag or a 10×His metal-binding sequence in place of the LBT.

### Expression and Purification of LBT-SELP Fusion Proteins

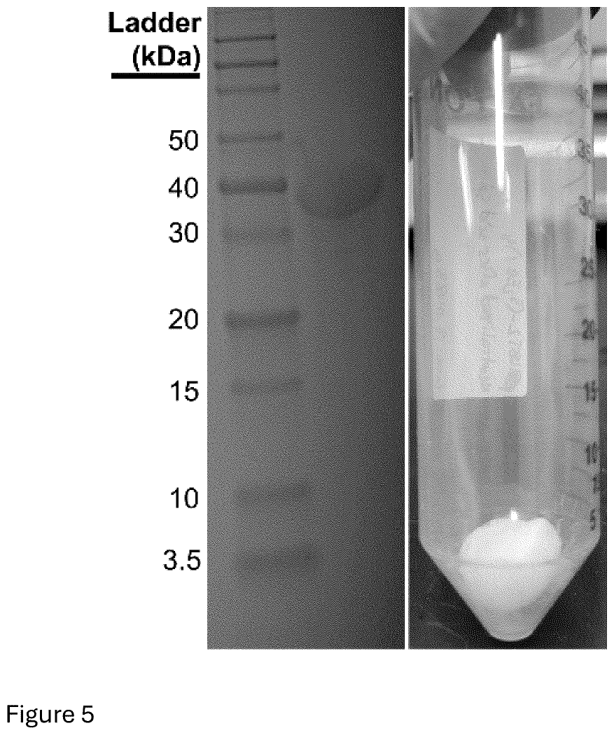

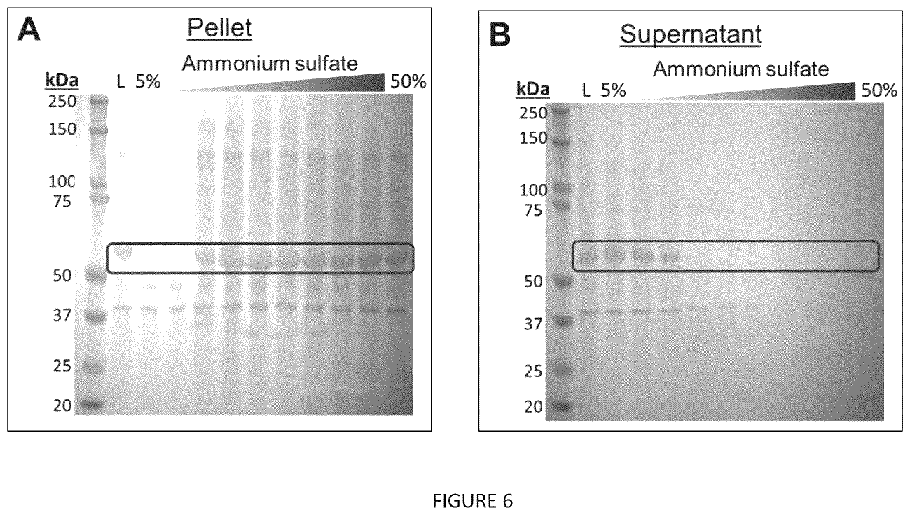

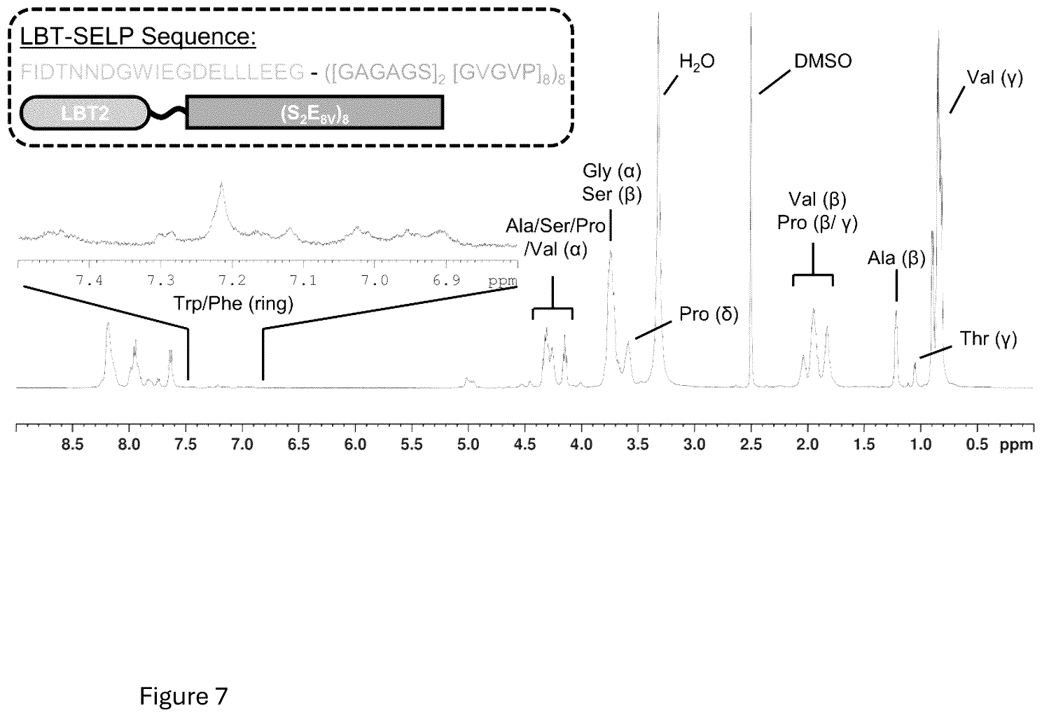

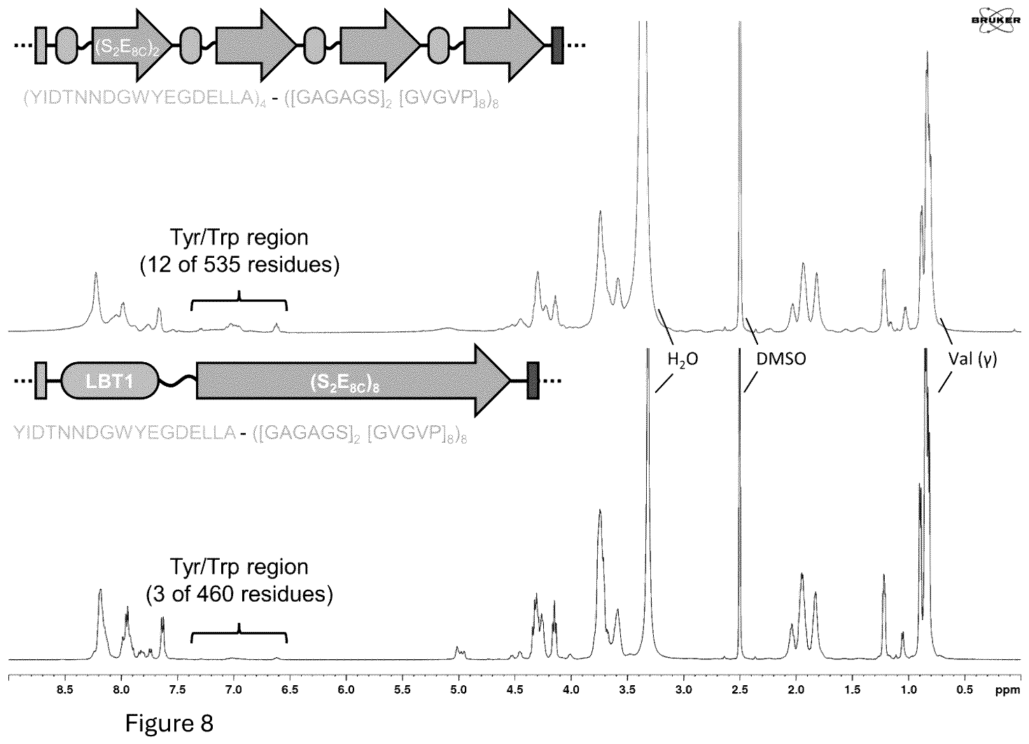

The creation of constructs for a series of LBT-SELP sequences allows the expression of LBT-SELP fusion proteins. In one example, the low-binding LBT-SELP fusion proteins were expressed in E. Coli and purified following traditional procedures (See Examples section). Purity was determined using SDS-PAGE by the presence of a single band as shown in FIG. 5 and FIG. 6. SELPs were further characterized by ¹H-NMR (Bruker Avance III, 500 Mhz) to verify the presence of the lanthanide binding sequence (FIG. 7-8).

### Solution-State Binding Assays of SELP-LBT Fusion Proteins

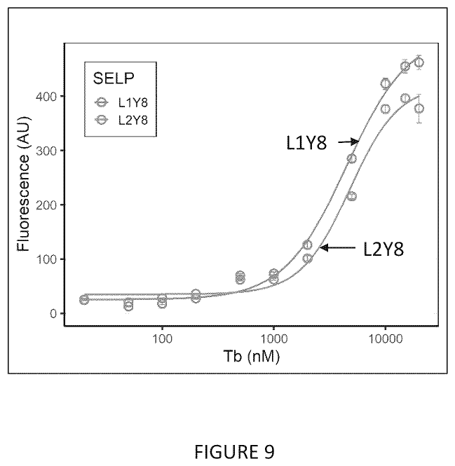

To efficiently recover metal ions from a solution, the LBT-SELPs must have high binding affinities for metal ions. LBTs with high native binding affinities, when incorporated with SELPS, can result in LBT-SELPs with high binding affinities for rare earth elements. The reported dissociation constants (K_{d}) of the LBT1 and LBT2 with respect to Tb³⁺, are reported as 57 nM (LBT1) and 19 nM (LBT2). In one example, the binding affinities of low-binding SELPs (L1Y8 and L2Y8, respectively) fusion proteins were determined by a titration experiment using L1Y8 (LBT1-S₂E_{8Y}) and L2Y8 (LBT2-linked S₂E_{8Y}). (FIG. 9) The apparent binding affinities (Kₐ) for L1Y8 and L2Y8 was determined to be 2.23×10⁵ M⁻¹ and 2.09×10⁵ M⁻¹, respectively.

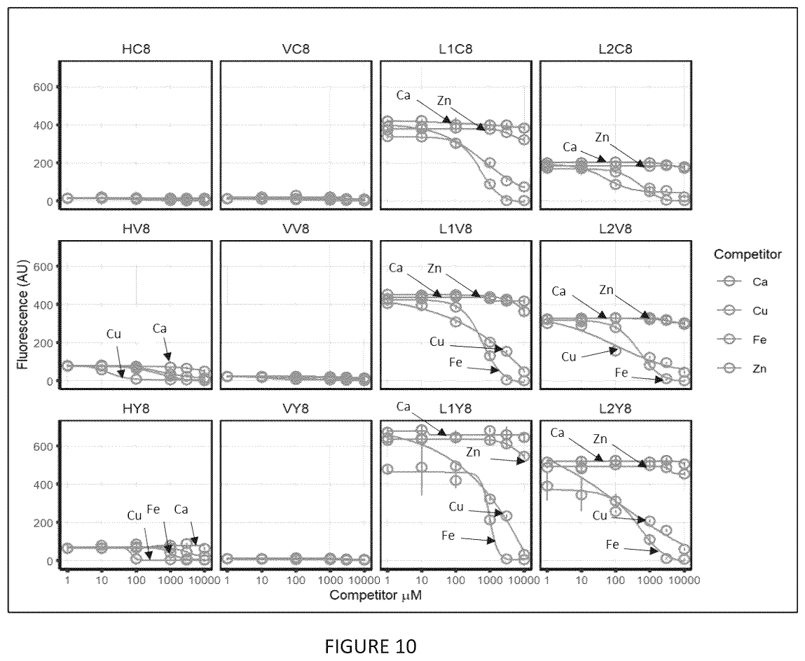

To efficiently recover metal ions from a solution containing other common metal ions, the LBT-SELP must be selective. In one example, a competition binding assay was performed to determine the specificity of low-binding SELPs to Tb³⁺ in the presence of four competitors (Ca²⁺, Cu²⁺, Fe³⁺, Zn²⁺) at a variety of concentrations. For all tested SELPs Ca²⁺ and Zn²⁺ had only minimal competitive effects however, Fe³⁺ and Cu²⁺ were both able to outcompete Tb³⁺ binding to a similar extent. (FIG. 10) It is contemplated that the binding affinity and selectivity of the LBT-SELPs can be tuned by choice of LBT.

### Preparation of SNF-based Membranes

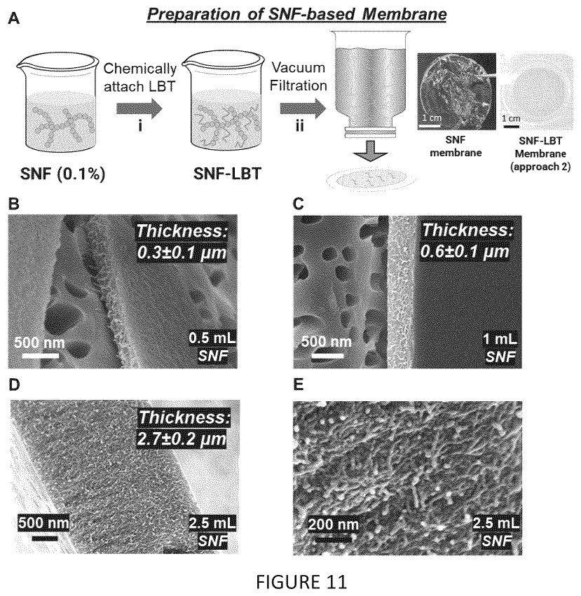

As shown in FIG. 11A, the SNF can be chemically functionalized with LBTs and then filtered over the substrate to form an SNF-LBT capture membrane. In brief, a volume of an SNF dispersion can be filtered over a porous substrate using vacuum filtration. The SNFs assemble and remain on top of the porous substrate. The formed SNF membrane thickness and pore size distribution are tunable by adjusting the initial volume of the SNF dispersion.

In one example, the SNF capture membrane thickness can be tuned by using different volumes of a SNF dispersion having a known amount of SNF. A larger volume of the dispersion results in a thicker capture membrane. FIGS. 11B-E are SEM images that show SNF capture membranes prepared by increasing the volume of SNF dispersion (0.1% w/v) have correspondingly increasing thicknesses.

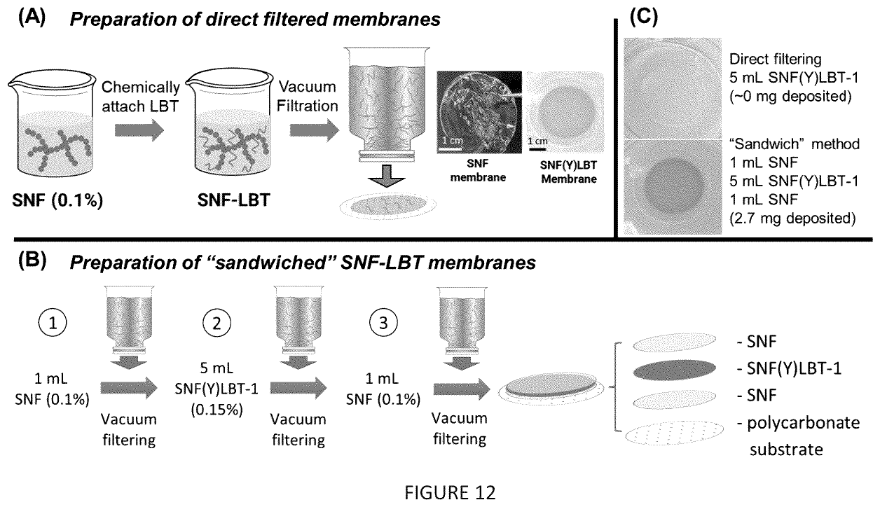

Forming capture membranes from the more heavily modified SNF(Y)LBT (with modified Asp, Glu, and Tyr residues) requires a different approach. The “sandwich” or multilayer method is shown in FIG. 12B. First, a solution of unmodified SNF can be used to form a first SNF membrane by filtration as described above. In a second step, a solution of modified SNF(Y)LBT can be added and vacuum filtered on top of the first SNF membrane. In a third step, an aliquot of a SNF solution can be applied with filtration to form a top layer to the membrane. This approach results in a multilayered capture membrane that successfully incorporates a layer of the functionalized SNF(Y)LBT between two layers of unmodified SNF membrane. (FIG. 12C). In addition, the sandwich method was demonstrated by depositing SELP-LBT-2 as a central layer. While the current example includes three steps, with two SNF layers and one SNF-LBT or SELP-LBT layer, it is contemplated that the steps could be repeated one or more times to produce a multilayered capture membrane.

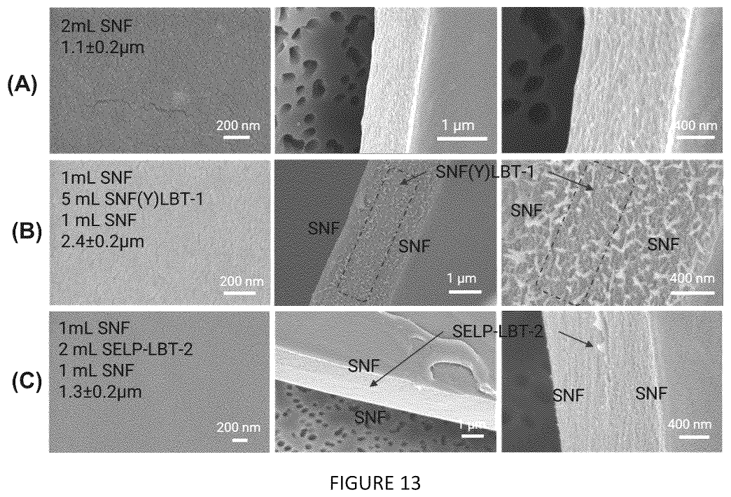

SNF capture membranes with variable thicknesses can be prepared using the sandwich method. SEM images in FIG. 13 show examples of capture membranes ranging from 1.3 to 2.4 μm in thickness. The nature of the silk component can affect the layer thickness. For example, a membrane of SNF(Y)LBT-1 (FIG. 13B), was significantly thicker than the control SNF membrane (FIG. 13A). A capture membrane with SELP-LBT-2 was prepared using the sandwich method as well. (FIG. 13C) The cross-section of the SNF membranes show the fibrillar morphology with nanosized pores is retained when the sandwich method is used.

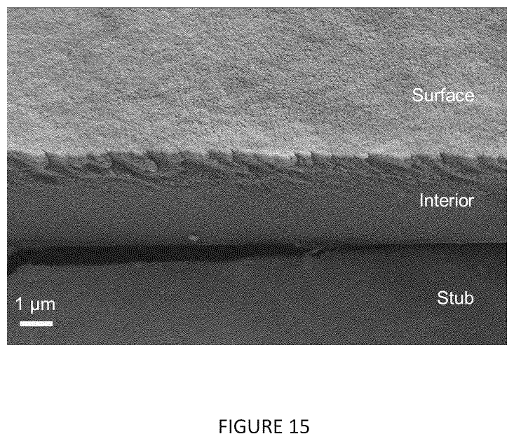

Deposition of the capture membrane can be enhanced by incubating the SNF dispersions with SELP fusion proteins before filtration. An increase in temperature and incubation time may result in increased beta-sheet formation between silk domains of the SELP and the SNF, thereby enhancing the deposition of SELP along with the SNF. In one example, SNF and SELP-LBT dispersions were incubated at 37° C. for 24 hours. An SEM cross-section of this self-assembled SNF-SELP capture membrane (24 hours, 37° C.) shows an increased average membrane thickness of 3±0.2 m. (FIG. 15).

### Analysis of SNF-SELP Hybrid Membranes for REE Capture

The SNF-SELP hybrid membranes can capture lanthanide ions from a solution that is passed through the membrane. In one example, a hybrid membrane containing SNF and the L2Y8 SELP captures 55-63% of Tb³⁺ in solution. For comparison, the SNF capture membrane having no LBT groups binds 23%-38% of available Tb³⁺, likely due to interactions with free acidic amino acid residues. Hybrid membranes generated from SNF-SELP solutions that were incubated for longer times did not capture as much Tb³⁺.

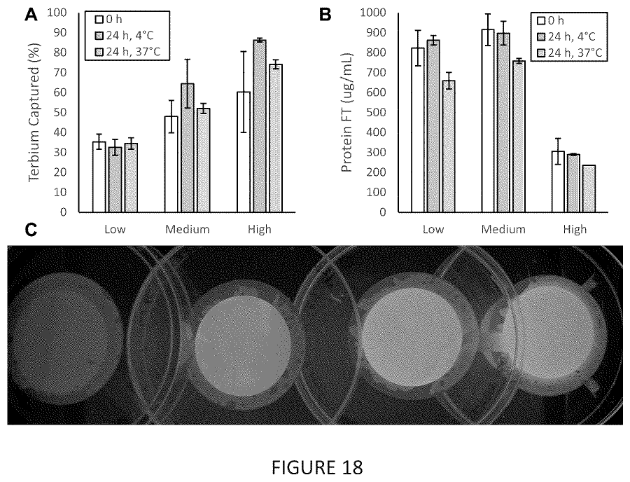

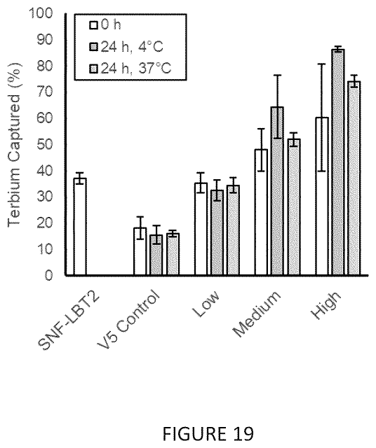

Increasing the number of LBT sequences in the SNF-SELPs used to make the hybrid membrane generally increases the amount of terbium captured, regardless of the incubation conditions. For example, membranes containing medium- and high-binding SELPs (LBT2₄-(S₂E_{8Y})₈ and LBT2₈-(S₂E_{8Y})₈, respectively) both capture more terbium than the membranes made with the low-binding SELP. (FIGS. 18AC, FIG. 19)

Increasing the number of LBT sequences in the SNF-SELPs used to make the hybrid membrane can increase the amount of SNF-SELP deposited in the membrane. For comparison, the amount of protein “flow through” was analyzed, that is, the portion of protein that flowed through the vacuum filtration and was not deposited in the membrane was analyzed. For both the low- and medium-binding SELPs, deposition of SELP was quite low (˜20% to 10%, respectively) while the high-binding SELPs deposited in much higher amounts (˜70%). (FIG. 18B)

### SELP Hydrogel Membranes

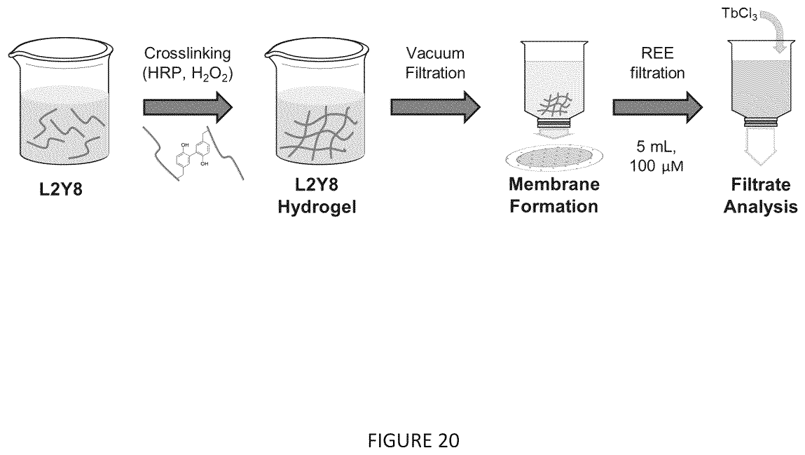

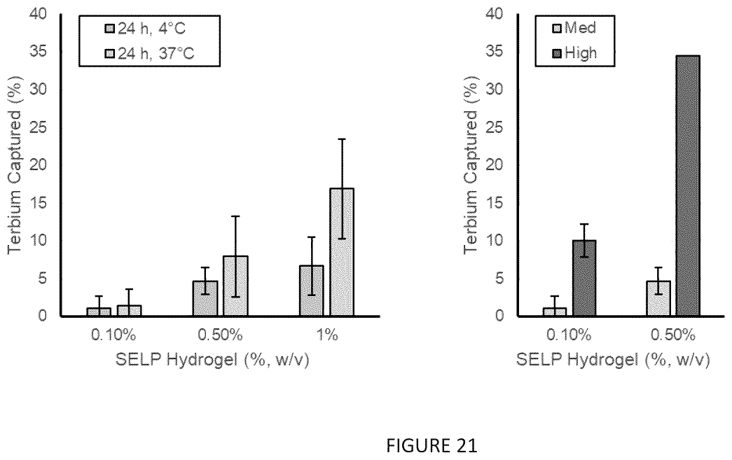

Hydrogel membranes can improve the amount of terbium captured. For example, a hydrogel membrane was prepared by cross-linking tyrosine-containing SELPs which were used to form a capture membrane by incubation with SNF solutions followed by filtration as described above. (FIG. 20). The hydrogel membrane made with the high-binding SELP this way demonstrated ability to capturing 6-10 times more terbium than membranes prepared with the medium binding SELP. (FIG. 21).

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Modification of Silk Nanofibrils

### Chemical Modification of Lanthanide Binding Tags (LBTs) on SNFs

As shown in FIGS. 1A and 1D, the chemical functionalization of SNFs can be achieved through Approach-1 utilizing existing carboxyl residues (˜1.1 mol %) from aspartic and glutamic acid and Approach-2 utilizing both tyrosine moieties (˜5.3 mol %) and the existing carboxyl residues in SNFs. The LBT functionalization of SNFs was conducted through direct carbodiimide coupling (approach-1) of LBT-1 (YIDTNNDGWYEGDELLA) or fluorescein isothiocyanate (FITC) tagged LBT-1 (YIDTNNDK(FITC)GWYEGDELLA) with the carboxyl residues in SNFs. The LBT functionalization was validated by fluorescence spectroscopy and microscopy of the SNF functionalized by LBT-1 (FITC).

Approach-2 targets maximizing the functionalization of LBT onto SNFs by increasing the amount of carboxyl residues in SNFs. This approach involves multiple steps: i) performing diazonium coupling at tyrosine moieties (˜5.3 mol %) of SNFs to increase carboxyl content; ii) carbodiimide coupling of LBTs to the existing carboxyl residue in SNFs and new carboxyl groups generated by diazonium coupling of the tyrosine residues. LBT-1 was functionalized onto SNFs through both approach-1 and approach-2.

Functionalization of SNFs with LBT-2 (FIDTNNDGWIEGDELLLEEG) was also completed through the direct carbodiimide coupling (approach-1). LBT-2 has a lower dissociation constant (19 nM) towards terbium than LBT-1, which suggests higher binding efficiency. he morphological studies through SEM showed that most SNFs retained their nanosized fibrillar structures after LBT functionalization by both approaches (FIG. 1.1 B, C, E, F).

### Quantification of Lanthanide Binding Tag Functionalization on SNFs

As shown in FIG. 2A, the chemical functionalization of SNFs with fluorescein isothiocyanate (FITC) tagged LBT-1 (YIDTNNDK(FITC)GWYEGDELLA) can be achieved by carbodiimide coupling with the carboxyl residues in SNFs (Asp and Glu). The LBT(FITC) functionalization was quantified by first generating a calibration curve with known concentrations of pure LBT(FITC) and then fitting the fluorescence of functionalized SNF-LBT(FITC) (FIG. 2B-D). Fluorescence was measured using a SpectraMax M2 microplate reader (Molecular Devices) using λₑₓ=490 nm and λₑₘ=520 nm, and the degree of functionalization was calculated to be ˜100% based on the concentration of LBT(FITC) compared to the theoretical maximum if all Asp and Glu residues were modified.

### Luminescence Spectroscopy of LBT-1 Functionalized SNF

The lanthanide binding behavior of LBT was investigated by luminescence spectroscopy. The lanthanides are sensitized by chelating with certain peptide structures through multiple side chains and have long-lived luminescence when excited under UV light. As shown in FIG. 3A, TbCl₃ and LBT-1 solutions at 100 μM exhibited negligible emission in the region of 470-630 nm, respectively. TbCl₃ solution in the presence of LBT-1 (1:1 molar ratio) showed four characteristic luminescence peaks (490, 545, 585, and 620 nm) corresponding to the different levels of electron transitions and associated photon emissions. The characteristic luminescence peaks of Tb³⁺ were clearly observed in the presence of LBT-1 functionalized SNFs through both approaches as shown in FIG. 3B. This observation verifies that the attached LBT is capable of chelating with lanthanides after functionalized onto SNFs. The LBT-1 functionalized SNFs by approach-2 exhibited marginally higher luminescence emission than SNFs functionalized by approach-1. The peak split between 530-560 nm suggests a slightly different complex structure between SNF(Y)-LBT-1 (functionalized by approach-2) with Tb³⁺. In the presence of unmodified SNF, Tb³⁺ exhibited similar luminescence emission patterns but at much lower intensity as the SNFs contain side chains (e.g., carboxyl and tryptophan moieties) that are also capable of chelating with lanthanides.

### Construction and Expression of LBT-SELP Fusion Constructs

A library of LBT-SELP expression vectors was developed with the completion of the set of “low-binding”, “medium-binding”, and “high-binding” constructs. These constructs that contain 2, 4 or 8 LBT sequences (LBT1 or LBT2) and 2, 4, or 8 SELP monomers (S₂E_{8C}, S₂E_{8V}, or S₂E_{8Y}) in an alternating fashion. In brief, the previously generated pET25-LBT1-(S₂E_{8X})₁ and pET25-LBT2-(S₂E_{8X})₁ constructs were digested with NheI and SpeI restriction enzymes and analyzed by agarose gel electrophoresis to identify the LBT-(S₂E_{8X})₁ fragments, which were excised and purified via DNA-binding columns and ligated into the original pET25-LBT1-(S₂E_{8X})₁ at the SpeI site to yield pET25-LBT1₂-(S₂E_{8X})₂. This concatemerization strategy was repeated twice more, generating pET25-LBT1₄-(S₂E_{8X})₄ and finally pET25-LBT1₈-(S₂E_{8X})₈ expression vectors.

### Expression and Purification of Low-Binding LBT-SELP Fusion Proteins

The low-binding LBT-SELP fusion proteins were expressed and purified in preparation for lanthanide binding assays. To express and purify these fusion proteins, chemically competent E. coli T7 Express (BL21 variant, New England BioLabs) was transformed with the respective expression vector constructed in the first quarter and colonies were isolated by selection on Luria broth (LB)-agar plates with ampicillin (100 mg/L). Successful transformants were cultured in 1 L of Terrific Broth (Fisher Scientific; 12 g/L casein peptone, 24 g/L yeast extract, 2.2 g/L monobasic potassium phosphate, 9.4 g/L dibasic potassium phosphate, 100 mg/L ampicillin) at 37° C. and 250 rpm reciprocal shaking, and protein expression was induced by the addition of isopropyl β-D-1-thiogalactopyranoside (IPTG, 1 mM final concentration) once they reached an optical density between 0.8 and 1.0. After 5 hours of growth post-IPTG induction, bacteria were harvested by centrifugation at 8,500 rpm and the pellet resuspended in 100 mL of phosphate buffered saline. Cells were lysed by sonication (15 min, 60% amplitude, 15 sec on/off cycles) and bacterial debris removed by centrifugation at 9500 rpm. The supernatant containing the LBT-SELP proteins was purified using inverse transition cycling (70° C. and 4° C.) as described by Huang, W., et al in “Design of Multistimuli Responsive Hydrogels Using Integrated Modeling and Genetically Engineered Silk-Elastin-Like Proteins,” Adv. Funct. Mater., 2016, 26(23), 4113˜4123; DOI: 10.1002/adfm.201600236. Purity was determined using SDS-PAGE by the presence of a single band as shown in FIG. 5, and pure LBT-SELPs were dried via lyophilization. Lyophilized SELPs were further characterized by ¹H-NMR (Bruker Avance III, 500 Mhz) to verify the presence of the lanthanide binding sequence (FIG. 7).

Lyophilized SELPs were further characterized by ¹H-NMR (Bruker Avance III, 500 Mhz) to verify the presence of the lanthanide binding sequence (FIG. 8). When compared to low-binding LBT-SELPs, an increase in signal intensity of ˜4-fold was observed in the aromatic region corresponding to the tyrosine and tryptophan residues present in the LBT sequence, which is to be expected

Construction and Expression of LBT-SELP Fusion Protein Constructs with Control Tags

The library of LBT-SELP expression vectors was expanded to include a set of control tags following the same low-, medium-, and high-binding pattern as the constructs designed previously (FIG. 4). Synthetic oligonucleotides encoding for two control tags, a 10×His metal-binding control and a non-metal binding V5 tag, were designed with two sets of flanking restriction enzymes, an upstream NdeI followed by NheI, and a downstream SpeI followed by SacI. The outer set of enzymes (NdeI and SacI) were used to clone the tags into a modified pET25 vector (pET25b[+] with the N-terminal His₆ tag removed). The 10×His replacement was chosen due to a closer size similarity to both V5 and the existing LBT tags, and an identical GGGGS linker was included in both tags immediately upstream of the SpeI cut site. The new vectors, pET25-His and pET25-V5, were used to construct the new sets of low-, medium-, and high-binding expression constructs using the concatemerization process outlined previously.

To express and purify medium-binding LBT-SELP fusion proteins, chemically competent E. coli T7 Express (BL21 variant, New England BioLabs) was transformed with the respective expression vector constructed in the first quarter and colonies were isolated by selection on Luria broth (LB)-agar plates with ampicillin (100 mg/L). Successful transformants were initially cultured in 1 L of Terrific Broth (Fisher Scientific; 12 g/L casein peptone, 24 g/L yeast extract, 2.2 g/L monobasic potassium phosphate, 9.4 g/L dibasic potassium phosphate, 100 mg/L ampicillin) at 37° C. and 250 rpm reciprocal shaking, and protein expression was induced by the addition of isopropyl β-D-1-thiogalactopyranoside (IPTG, 1 mM final concentration) once they reached an optical density between 0.8 and 1.0. After 5 hours of growth post-IPTG induction, bacteria were harvested by centrifugation at 8,500 rpm and the pellet resuspended in 100 mL of phosphate buffered saline. Cells were lysed by sonication (15 min, 60% amplitude, 15 sec on/off cycles) and bacterial debris removed by centrifugation at 9500 rpm. The supernatant containing the LBT-SELP proteins was purified using inverse transition cycling (70° C. and 4° C.) as described by Huang, W., et al. However, difficulties arose during the inverse transition cycling with the formation of insoluble precipitates, believed to be the product of irreversible 3-sheet formation, and purification of these medium-binding SELPs could not be completed.

To address this issue, decreasing the high temperature of the inverse-transition cycle to either 42° C. or 55° C. However, neither of these temperatures were sufficient to precipitate the medium-binding LBT-SELPs. Addition of additional NaCl (to 3% final concentration) did induce precipitation at both temperatures, however the insolubility issue remained once they precipitated. The inverse-transition cycling was replaced with traditional ammonium sulfate precipitation using a saturated ammonium sulfate solution at 4° C. Fractions of crude cell lysate containing medium-binding SELP LBT1₄(S₂E_{8C})₈ were mixed with saturated ammonium sulfate at final concentrations ranging from 5% to 50% saturation (in 5% increments), and the fractions centrifuged at 14,000 rpm and 4° C. for 5 minutes to pellet any precipitate. Both pellets and supernatant were analyzed by SDS-PAGE as shown in FIG. 7. Based on this data, a final concentration of 20% was chosen for the remainder of the cell lysate, as it resulted in the complete precipitation of the target medium-binding proteins from the supernatant (FIG. 7B). The pelleted protein was successfully resuspended in cold DI H₂O after 30 minutes of mixing, while several impurities remained insoluble and were separated by centrifugation at 9500 rpm (4° C., 20 min). Repeating this cold precipitation/dissolution step further reduced impurities to below detectable levels in most cases, and purified proteins were dialyzed against DI H₂O to remove excess salts (with a molecular weight cut-off of 10 kDa). The successfully purified LBT1₄(S₂E_{8C})₈ was lyophilized for further characterization.

### Solution-State Binding Assays of SELP-LBT Fusion Proteins

In order to determine the binding affinity of LBT-SELP fusion proteins, a titration experiment was performed using LBT1- and LBT2-linked S₂E_{8Y} low-binding SELPs (L1Y8 and L2Y8, respectively). A protein concentration of 10 μM was used, and TbCl₃ concentration was varied from 20 to 20000 nM with 4 replicates per titration. SELPs were mixed with TbCl₃ solutions in a 96-well microtiter plate and incubated at 4° C. for 20 min prior to fluorescence measurements. Fluorescence was measured using a SpectraMax M2 microplate reader (Molecular Devices) using λₑₓ=280 nm and λₑₘ=544 nm, and the data is shown in FIG. 9. Titration data was analyzed by log-logistic regression and binding affinity (1/K_{d}) values were determined using the drc (dose response curve) package in R. The apparent binding affinity (Kₐ) for L1Y8 and L2Y8 was determined to be 2.23×10⁵ M⁻¹ and 2.09×10⁵ M⁻¹, respectively. However, this apparent Kₐ is likely to be lower than the true binding affinity which would need to be ascertained using a SELP concentration ≈ the dissociation constant (K_{d}) of the LBT moieties with respect to Tb³⁺, which literature reports as 57 nM (LBT1) and 19 nM (LBT2), with multiple Tb³⁺ concentrations both above and below this concentration.

A competition binding assay was performed to determine the specificity of low-binding SELPs to Tb³⁺ in the presence of other metal competitors. Tb³⁺ concentrations were kept constant at 10 μM, and mixed with one of the four competitors (Ca²⁺, Cu²⁺, Fe³⁺, Zn²⁺) at a variety of concentrations from 0 to 10000 μM and allowed to incubate for at least 20 minutes at 4° C. These mixed Tb/competitor solutions were then combined with each SELP (prepared at a 10 μM concentration) and allowed to equilibrate for 20 minutes at 4° C. before measuring the fluorescence as described above. The data was plotted and analyzed by log-logistic regression using the drc (dose response curve) package in R (FIG. 10).

Ca²⁺ and Zn²⁺ had only minimal competitive effects for all tested SELPs, even at a 1000× higher concentration than Tb³⁺. However, Fe³⁺ and Cu²⁺ were both able to outcompete Tb³⁺ binding to a similar extent, reducing the fluorescence intensity by 54˜77% at 100× concentrations and ˜100% at 1000× concentrations, with no significant differences noted between the three SELP sequences.

### Preparation of SNF-Based Membranes

The SNF membranes were prepared according to the following protocol. A SNF dispersion in water is prepared and a volume of the SNF dispersion is filtered over a porous polycarbonate membrane substrate (pore size: 0.2 μm) using vacuum filtration. (FIG. 11A) Both standalone SNF membranes and LBT functionalized SNF membranes can be prepared by this method. In FIG. 11B-E, the SEM images showed that SNF membranes with thicknesses ranging from 0.3 to 2.7 μm can be prepared by adjusting the SNF dispersion (0.1% w/v) volume between 0.5 mL and 2.5 mL. The cross-section of the SNF membranes exhibited clear fibrillar morphology with nanosized pores. Both stand alone SNF membranes and LBT functionalized SNF membranes can be prepared by this method. (FIG. 12A)

Forming membranes using the more heavily modified SNF(Y)LBT (with modified Asp, Glu, and Tyr residues) requires a different approach as shown in. The “sandwich” method is shown in FIG. 12B. 1 mL of unmodified SNF solution (0.1%) was first used to form a membrane by direct vacuum filtration. Next, 5 mL of modified SNF(Y)LBT solution (0.15%) was vacuum filtered on top of the first SNF membrane. Finally, 1 mL of SNF solution (0.1%) was applied to form a top layer to the membrane. This approach resulted in a multilayered membrane that successfully incorporated the functionalized SNF(Y)LBT, although only 10% of the applied solution was deposited as determined gravimetrically (FIG. 12C). In addition, the sandwich method was used to deposit SELP-LBT-2 as a central layer in a preliminary experiment with materials from the bioengineering approach.

In FIG. 13A-C, SEM images show that multilayered SNF membranes with thicknesses ranging from 1.3 to 2.4 μm can be prepared using the sandwich method. The cross-section of the SNF membranes exhibited clear fibrillar morphology with nanosized pores. Despite the low mass of SNF(Y)LBT-1 deposited (FIG. 13B), this membrane was significantly thicker than the control SNF membrane formed after two consecutive applications of 1 mL SNF solution (FIG. 13A). No apparent difference in morphology was observed between the layers of this multilayered membrane. In contrast, the SELP-LBT-2 multilayered membrane (FIG. 13C) showed only a thin deposition layer of ˜0.2 μm.

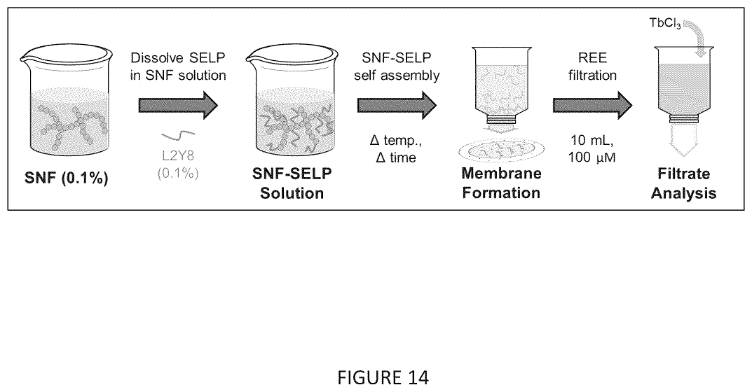

Using the “sandwich” method, several multilayered membranes were prepared consisting of an upper and lower layer of SNF with a layer of SELP-LBT in between. However, there can be low deposition of the SELP component (<10% of applied SELP deposited). To improve upon the membrane design, a new strategy was devised to exploit the self-assembling nature of SELPs to make hybrid membranes containing a mixture of SNF and LBT-SELPs in the membrane layer. (FIG. 14) In one example, L2Y8 (LBT2-(S₂E_{8Y})₈) was dissolved in a solution of SNF (0.1% w/v) at an equal concentration (0.1%) with 30 minutes of mixing at 4° C. This SNF-SELP solution was divided into aliquots and each aliquot was subjected to a different combination of self-assembly conditions varying time and temperature. The SNF-SELP solutions were incubated at either 4° C. or 37° C., for either 24 hours or 7 days. After incubation, each aliquot was subjected to vacuum filtration to form a hybrid membrane. A representative SEM cross-section of self-assembled SNF-SELP hybrid membrane (24 hours, 37° C.) showed an average membrane thickness of 3±0.2 μm. (FIG. 15) It is contemplated that SELP-LBTs can be combined with LBT-modified SNFs to increase the number of metal binding sites in another example of a hybrid membrane.

### Analysis of SNF-SELP Hybrid Membranes for REE Capture

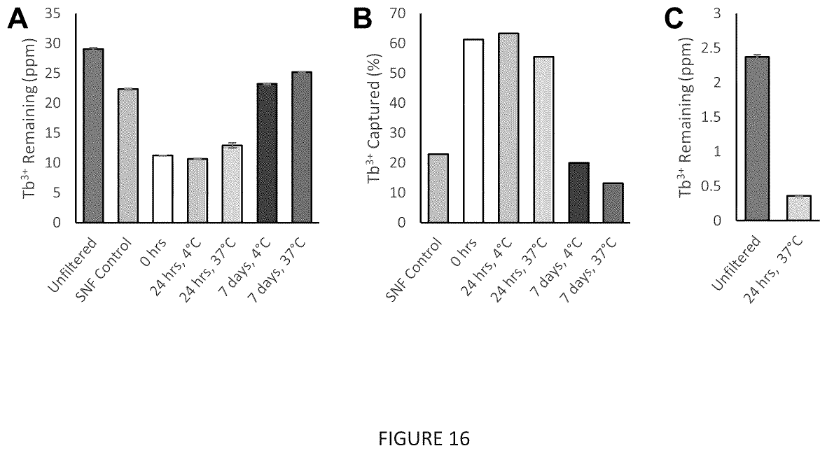

To analyze the metal ion capture ability of the hybrid membranes, the hybrid membranes were used immediately after formation to filter 10 mL of a 100 μM solution of TbCl₃. The filtrate was analyzed by inductively-coupled plasma optical emission spectroscopy (ICP-OES 5100, Agilent Technologies) to determine the Tb³⁺ concentration before and after filtration (FIGS. 16A and B). The SNF alone was capable of binding 23% of available Tb³, and the inclusion of L2Y8 increased the binding capacity to 55-63% for the 24-hour assemblies as well as the 0-hour control. Continued incubation had a detrimental effect on the binding capacity.

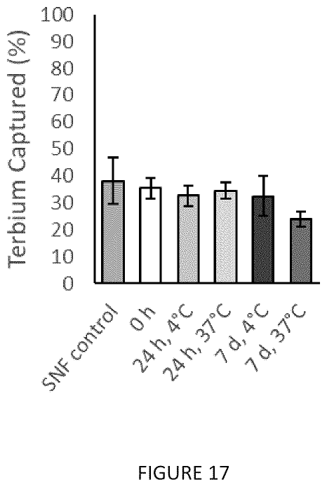

In another example, LBT2-(S₂E_{8Y})₈ was dissolved in a solution of SNF (0.1% w/v) at an equal concentration (0.1%) with 30 minutes of mixing at 4° C. This SNF-SELP solution was divided into several aliquots and subjected to different self-assembly conditions; they were incubated at either 4° C. or 37° C., for either 24 hours or 7 days. The hypothesis for these experiments was that an increase in temperature and incubation time would result in increased beta-sheet formation between silk domains of the SELP and the SNF, thereby enhancing the deposition of SELP along with the SNF. Each aliquot was subjected to vacuum filtration to form the membranes (and the protein flow-through saved for later analysis), then used immediately to filter 5 mL of a 100 μM solution of TbCl₃. The filtrate was analyzed by inductively-coupled plasma optical emission spectroscopy (ICP-OES 5100, Agilent Technologies) to determine the Tb³⁺ concentration before and after filtration (FIG. 17). In this case, the SNF alone was capable of binding ˜38% of available Tb³⁺, likely due to non-specific adsorption onto acidic amino acid residues, however the inclusion of L2Y8 had no significant effect on the binding of terbium (single factor ANOVA, α=0.05). Although it was not found to be significant, likely due to the high variance observed for all groups, the 7-day incubation at 37° C. did appear to have a negative effect on terbium capture in agreement with earlier reports.

Hybrid membranes were formed with the medium- and high-binding variants of this SELP (LBT2₄-(S₂E_{8Y})₈ and LBT2₈(S₂E_{8Y})₈, respectively). As shown in FIG. 18AC, increasing the number of lanthanide binding tags increased the amount of terbium captured regardless of the incubation conditions (with the exception of high-binding SELP at 0 h).

To investigate the amount of SELP deposited within the membranes, the protein flow-through after membrane formation was saved and analyzed using the BCA assay in 96-well microtiter plates, according to the manufacturers protocol (Thermo Scientific). The nanofibrils were ignored for this assay as they are too large to pass through the support filter (0.2 μm polycarbonate) and make up the majority of the membrane as observed by SEM, so it is assumed that SELP is the only protein component in the flow-through. The data is shown in FIG. 18B, measured as the concentration of protein remaining in the flow-through (FT) expressed in g/mL. For both the low- and medium-binding SELPs, deposition of SELP was quite low (˜20% to 10%, respectively). The high-binding SELPs deposited in much higher amounts (˜70%), however this was confounded by the presence of a large portion of insoluble material when the SELPs were first dissolved. Interestingly, the 24 h incubation at 37° C. did increase the amount of protein deposited, although from the ICP-OES data it appears to have had no effect or a detrimental effect on the capture of terbium.

### Analysis of SNF-LBT2 Membranes for REE Capture

SNF was directly conjugated with LBT2 (SNF-LBT2) using carbodiimide coupling and SNF-LBT2 membranes were prepared similarly to the hybrid SNF-SELP membranes by applying 5 mL of 0.1% SNF-LBT2 solution onto a polycarbonate (PC) support (0.2 μm pore size). The solution was vacuum filtered to deposit it onto the polycarbonate support, washed with 5 mL of DI water, and then immediately used to filter 5 mL of 100 μM TbCl₃ solution in acetate buffer (5 mM, pH 4). Flow-through was collected and analyzed by inductively-coupled plasma optical emission spectroscopy (ICP-OES 5100, Agilent Technologies) to determine residual terbium concentration (FIG. 19). Additionally, a non-metal binding control, V5-(S₂E_{8Y})₈, was prepared as a hybrid membrane by dissolving in a solution of SNF (0.1% w/v) at an equal concentration (0.1%) with 30 minutes of mixing at 4° C. The SNF-SELP solution was either used to form membranes immediately (0 h), or incubated for 24 h at either 4° C. or 37° C. as done with previous experiments, and terbium capture was analyzed by ICP-OES (FIG. 19). As expected, the V5-tagged control SELPs captured the lowest amount of terbium, although this one did capture ˜16% of the available terbium. This is likely due to non-specific adsorption onto the surface of the SNFs. The chemically derived SNF-LBT2 membranes performed similarly to the low-binding SNF-L2Y8 membranes, with no significant differences observed (single factor ANOVA, α=0.05).

### SELP Hydrogel Membranes

Initial experiments were performed with medium-binding SELP L2₄Y₈, which was dissolved in cold DI water for 30 min at 4° C. with gentle mixing, at a final concentration of either 0.1%, 0.5%, or 1.0% (w/v). HRP was added to a final concentration of 10 units/mL, followed by H₂O₂ at a final concentration of 0.1% (v/v). The solutions were mixed by vortex and incubated for 24 h at either 4° C. or 37° C. and were vacuum filtered onto PC support membranes and used immediately for terbium filtration as described above. One difference with these experiments is that the support membrane and vacuum filtration apparatus was scaled down to ⅕^{th} the size of previous experiments to reduce the mass of SELP and allow for more replicates (n=6). To maintain a similar ratio of protein per membrane, the volume of SNF-SELP solution was also reduced to ⅕ (1 mL); however, to maintain a terbium concentration above the limit of detection for ICP-OES the volume (5 mL) and concentration (100 μM) of the TbCl₃ solution remained the same.

The results of the medium-binding L2₄Y₈ SELPs are shown in FIG. 20A. The temperature of the incubation had a large effect on the amount of terbium captured, which was significant for the highest concentration of SELP used (1%, one-way ANOVA, α=0.05). A trend of increasing terbium capture with increasing SELP concentration was observed, which was significant between the 0.1% and 1.0% sample groups.

To compare the effect of tag number using this hydrogel membrane technique, membranes were also formed using the high-binding L2₈Y₈ SELPs with a 24 h incubation at 37° C. The high-binding SELPs were capable of capturing 6-10 times more terbium than the medium binding (FIG. 20B). However, there was a drastic reduction in flux through the 0.5% membranes, likely due to a large amount of protein deposition coupled with the smaller pore sizes of a cross-linked hydrogel. This resulted in clogged membranes for all but one replicate. Due to this issue, experiments planned with 1% high-binding SELP were abandoned.

1. A recombinant fusion protein comprising at least one lanthanide metal binding sequence (SEQ ID NO: 1-4) covalently bound to a silk-elastin-like polymer (SELP), wherein the recombinant fusion protein exhibits an apparent or true binding affinity towards a lanthanide metal ion of between 2.0×10⁵ M⁻¹ and 1.8×10⁷ M⁻¹ or higher.

2. The recombinant fusion protein of claim 1, wherein the SELP comprises the sequence ((GAGAGS)ₐ(GXGVP)_{b})_{c}, wherein a ratio of a to b is between 1:3 and 1:5, wherein a is 1, 2, or 3, wherein c is between 5 and 20, and wherein X is valine, cysteine, or tyrosine.

3. The recombinant fusion protein of claim 1, wherein the at least one lanthanide metal binding sequence terminates the recombinant fusion protein.

4. The recombinant fusion protein of claim 1, wherein the at least one lanthanide metal binding sequence repeats in an alternating pattern with SELP sequences.

5. A modified silk nanofibril wherein a lanthanide metal binding molecule is coupled to a surface of a silk nanofibril, wherein the lanthanide metal binding molecule comprises at least one lanthanide metal binding sequence (SEQ ID NO: 1-4).

6. A capture membrane comprising at least one layer comprising the recombinant fusion protein of claim 1.

7. A multilayered membrane comprising at least a first layer, a second layer, and a top layer, wherein the first, second, or top layer comprises the recombinant fusion protein according to claim 1, wherein the first layer does not include a metal-binding protein, and wherein the second layer is situated between the first layer and the top layer.

8. The multilayered membrane of claim 7, wherein a thickness of the membrane is between 0.2 μm and 3.2 μm.

9. The multilayered membrane of claim 7, wherein the first, second, or top layer is porous, with pore diameters between 10 and 50 nm.

10. A hydrogel membrane comprising a plurality of the recombinant fusion protein of claim 1, wherein recombinant fusion proteins of the plurality of recombinant fusion proteins are covalently crosslinked with one another.

11. The hydrogel membrane of claim 10 wherein the recombinant fusion proteins are crosslinked between tyrosine residues.

12. A method of producing the recombinant fusion protein of claim 1, comprising:
(i) expressing in a host cell the recombinant fusion protein comprising at least one metal-binding sequence and at least one SELP sequence; wherein the at least one metal-binding sequence is one of SEQ ID NO: 1-4; and
(ii) purifying the recombinant fusion protein.

13. A modified silk protein comprising:
(i) at least one modified lanthanide metal-binding protein according to SEQ ID NO: 1-4, or a variant thereof which differs in each case from said sequence by 1 amino acid substitution, 1 to 2 amino acid deletion(s), and/or 1 to 2 amino acid insertion(s); and
(ii) at least one modified silk-elastin-like polymer (SELP), wherein the SELP comprises the sequence ((GAGAGS)ₐ(GXGVP)_{b})_{c}, wherein a ratio of a to b is between 1:3 and 1:5, wherein a is 1, 2, or 3, wherein c is between 5 and 20, and wherein X is valine, cysteine, or tyrosine, or a variant thereof which differs in each case from said sequence by 1 amino acid substitution, 1 to 2 amino acid deletion(s), and/or 1 to 2 amino acid insertion(s).

14. A method of creating a layered membrane according to the following steps:
(i) a first dispersion of a silk nanofibril is filtered over a porous polycarbonate membrane substrate using vacuum filtration to form a first layer; and
(ii) a second dispersion of the recombinant fusion protein of claim 1 is vacuum filtered over the first layer to create a second layer;
wherein the first dispersion and second dispersion each have a protein concentration of between 0.1% and 1.0%, weight by volume.

15. The method of claim 14, wherein the steps (i) and (ii) are repeated between 1 and 10 times followed by step (i) as a final step.

16. A method of detecting a lanthanide metal ion species in solution using the recombinant fusion protein of claim 1, wherein the recombinant fusion protein is added to a solution and the solution is interrogated spectroscopically for a characteristic of the at least one lanthanide metal binding sequence chelated to the lanthanide metal ion species.

17. The method of claim 16, wherein the characteristic is an emission.

18. The method of claim 16, wherein the characteristic is an absorption.

19. A hybrid membrane comprising a mixture of silk nanofibrils and the recombinant fusion protein of claim 1, wherein the mixture of silk nanofibrils and the recombinant fusion protein are combined in solution and deposited by filtration on a support to form the hybrid membrane.

20. A method of removing lanthanide metal ions from a solution comprising the following steps:
(i) the recombinant fusion protein of claim 1 is dissolved and the solution is incubated between 0 and 24 hours between 4° C. and 37° C. with a solution of silk nanofibrils to form a combined fusion protein silk nanofibril solution;
(ii) the combined fusion protein silk nanofibril solution is used to form at least one hybrid membrane by vacuum filtration over a porous support; and
(iii) a solution containing lanthanide metal ions is filtered through the at least one hybrid membrane.
